(19) United States
(12) Patent Application Publication
(10) Pub. No.: US 2025/0250604 A1
(43) Pub. Date: Aug. 7, 2025

(54) METHODS FOR PRODUCTION OF ASTAXANTHIN AND RELATED ANALOGS

(71) Applicant: BIGELOW LABORATORY FOR OCEAN SCIENCES, East Boothbay, ME (US)

(72) Inventors: Michael W. Lomas, Damariscotta, ME (US); Benjamin Wheeler, Trevett, ME (US); Robert Schmedicke, Boothbay Harbor, ME (US); Michael Preston, Alna, ME (US)

(21) Appl. No.: 19/035,548

(22) Filed: Jan. 23, 2025

Related U.S. Application Data
(60) Provisional application No. 63/623,989, filed on Jan. 23, 2024.

(51) Int. Cl. C12P 23/00; A61K 8/35; A61K 31/122; A61Q 19/00; C12M 1/00; C12M 3/00; C12P 5/00

(52) U.S. Cl. CPC C12P 23/00; A61K 8/35; A61K 31/122; A61Q 19/00; C12M 21/02; C12M 23/44; C12P 5/007

(57) ABSTRACT

The present disclosure features methods of producing and accumulating a compound of Formula (I) and related analogs, e.g., in an algal cell.

### CLAIM OF PRIORITY

This application claims priority to U.S. Application No. 63/623,989, filed Jan. 23, 2024; the entire contents of the foregoing application are hereby incorporated herein by reference.

### FIELD OF TECHNOLOGY

The disclosure relates to methods of production and accumulation of astaxanthin and related analogs. More particularly, the disclosure relates to methods of production and accumulation of astaxanthin and related analogs by culturing algal cells.

### SUMMARY

The present disclosure features methods of producing and accumulating a compound of Formula (I), e.g., astaxanthin and related analogs, e.g., in an algal cell. In one aspect, the method comprises providing the algal cell in a red motile or growth phase. In an embodiment, the method further comprises culturing the algal cell in a cell culture medium under conditions (i) sufficient to increase production and/or accumulation of astaxanthin in the algal cell; or (ii) sufficient to delay transition of the algal cell from the red motile/growth phase to the cyst phase. In an embodiment, the amount of astaxanthin or a related analog produced is between about 1-10% w/w of total biomass (e.g., between 1-9, 1-8, 1-7, 1-6, 1-5, 1-4, 1-3, 1-2% w/w total biomass). In an embodiment, the amount of astaxanthin or a related analog produced is between about 1-5% w/w of total biomass.

In an embodiment, the total biomass comprises total cell mass of algal cells in the red motile or growth phase. In an embodiment, the algal cells in the red motile/growth phase include cells in a palmelloid phase. In an embodiment, the amount of astaxanthin or a related analog produced is at least about 25%, 50%, 75%, or 100% greater than an amount of astaxanthin produced by a reference standard, e.g., the amount of astaxanthin or a related analog produced under conditions that do not delay transition of the algal cell from the red motile/growth phase to the cyst phase. In an embodiment, the duration of the culturing of the algal cell in the red motile/growth phase is between 2-10 days, e.g., between 3-8 days, between 4-7 days, e.g., about 2, 3, 4, 5, 6, 7, 8, 9, or 10 days for a cell culture of about 7,000 algal cells/mL.

In an embodiment, the delay of the transition of the algal cell from the red motile/growth phase to the cyst phase lasts for 2-14 days, e.g., between 5-14 days, between 7-14 days, e.g., about 2, 3, 4, 5, 6, 7, 8, 9, 10, 11, 12, 13, or 14 days. In an embodiment, the method further comprises culturing the algal cell in a green motile/growth phase. In an embodiment, the algal cell in the green motile/growth cell is present in a cell culture medium having depleted nitrate, e.g., less than 10 mg/L, 8 mg/L, 6 mg/L, 5 mg/L, 4 mg/L, 3 mg/L, 2 mg/L, or 1 mg/L. In an embodiment, the method further comprises culturing the algal cell in a green motile/growth phase under an irradiance of between 5-fold to 15-fold, e.g., between 8-fold to 12-fold, e.g., 8-fold, 9-fold, 10-fold, 11-fold, or 12-fold, over a reference standard, e.g., the irradiance under conditions that do not increase production and/or accumulation of astaxanthin in the algal cell. In an embodiment, the cell culture medium comprises a plurality of trace metals (e.g., zinc or vanadium). In an embodiment, the cell culture medium comprises one or more of Na₃VO₄, ZnSO₄, MnCl₂, CoCl₂, Na₂MoO₄, H₂SeO₃, and FeCl₃.

In an embodiment, the cell culture medium is effective to increase growth rate of the algal cells cultured in a green motile/growth phase by at least 0.5-fold, e.g., 1-fold, 1.5-fold, 2-fold, 2.5-fold, or 3-fold, as compared to a reference standard, e.g., a reference cell culture medium, e.g., a cell culture medium comprising copper and substantially free of zinc and vanadium trace metals. In an embodiment, the cell culture medium is effective to increase cell biomass yield of algal cells cultured in the green motile/growth phase by at least 1.5-fold, 2-fold, 2.5-fold, or 3-fold, as compared to a reference standard, e.g., a reference cell culture medium, e.g., a cell culture medium comprising copper and being substantially free of zinc and vanadium trace metals.

In an embodiment, the cell culture medium is effective to produce a growth rate of the algal cells between about 0.2-0.5 d⁻¹, e.g., 0.3-0.4 d⁻¹. In an embodiment, the cell culture medium is effective to produce a cell biomass yield of algal cells cultured in the green motile/growth phase of between about 0.2-0.5 g 1⁻¹ dry weight (DW), e.g., 0.3-0.4 g 1⁻¹. In an embodiment, the cell culture medium is effective to reduce, limit, or inhibit production of intracellular reactive oxygen species (ROS). In an embodiment, the cell culture medium comprises nitrate and phosphorus in a ratio of less than 19:1 (N:P), e.g., less than 18:1, 15:1, 12:1, 11:1, 10:1, 9:1, 8:1, 7:1, 6:1, 5:1, 4:1, 3:1, or 2:1. In an embodiment, the cell culture medium comprises nitrate and phosphorus in a ratio of less than 12:1 (N:P). In an embodiment, the cell culture medium comprises nitrate and phosphorous in a ratio of between about 11:1 (N:P) and 5:1 (N:P).

In an embodiment, the cell culture medium is effective to delay transition of at least about 70%, e.g., 80%, 90%, or 99% of the algal cells from the red motile/growth phase to the cyst phase after 6 days of culturing the algal cell in the red motile/growth phase. In an embodiment, the cell culture medium is effective to delay transition of at least about 50%, e.g., 60%, 70%, 80%, 90%, or 99% of the algal cells from the red motile/growth phase to the cyst phase after 12 days of culturing the algal cell in the red motile/growth phase.

In an embodiment, the method further comprises controlling temperature of the culture to between about 15-28° C., e.g., 18-26° C. or 20-24° C. In an embodiment, the method further comprises culturing the algal cell under light intensity at an irradiance between about 150-1400 μmol photons m⁻² s⁻¹, e.g., 175-1100 μmol photons m⁻² s⁻¹ or 200-800 μmol photons m⁻² s⁻¹. In an embodiment, the method further comprises culturing the algal cell under light intensity at an irradiance between about 30-140 μmol photons m⁻² s⁻¹, e.g., 35-110 μmol photons m⁻² s⁻¹ or 40-80 μmol photons m⁻² s⁻¹. In an embodiment, the method further comprises culturing the algal cell in light/dark conditions at a ratio of between about 9:15 to 15:9 (L:D), e.g., 9:15 to 10:14 (L:D), 10:14 to 13:11 (L:D), or 13:11 to 15:9 (L:D). In an embodiment, the method further comprises controlling pH of the culture to between about 6-8, e.g., 6, 6.5, 7, 7.5, or 8.

In an embodiment, the method further comprises culturing the algal cell at an initial cell density of between about 1×10³ and 1×10⁶ cells ml⁻¹, e.g., about 1×10³, 1×10⁴, 1×10⁵, or 1×10⁶ cells ml⁻¹. In an embodiment, the method further comprises culturing the algal cell at an initial cell abundance of between about 2×10⁴ and 12×10⁴ cells ml⁻¹, e.g., 2×10⁴, 3×10⁴, 4×10⁴, 5×10⁴, 6×10⁴, 8×10⁴, 10×10⁴, or 12×10⁴ cells ml⁻¹. In an embodiment, the method further comprises culturing the algal cell under controlled carbon dioxide (CO₂) conditions, e.g., between about 1-5% CO₂, e.g., 1-2% CO₂, 1% CO₂, 2% CO₂, 3% CO₂, 4% CO₂, or 5% CO₂. In an embodiment, the method further comprises separating/collecting the algal cell in the red motile/growth phase.

In an embodiment, the algal cell is a Haematococcus cell. In an embodiment, the algal cell is selected from the group consisting of Haematococcus capensis, Haematococcus carocellus, Haematococcus droebakensis, Haematococcus lacustris, Haematococcus murorum, Haematococcus pluvialis, Haematococcus thermalis, and Haematococcus zimbabwiensis.

In an embodiment, the method further comprises providing the algal cell in the green motile/growth phase. In an embodiment, the method further comprises culturing the algal cell in a cell culture medium under conditions that allow transition of the algal cell from the green motile/growth phase to the red motile/growth phase. In an embodiment, the method further comprises separating/collecting the algal cell in the red motile/growth phase to provide the algal cell in the red motile/growth phase for production of the astaxanthin or related analog.

In another aspect, the present disclosure features an algal cell in the red motile/growth phase cultured by the methods described herein. In an embodiment, the algal cell comprises an effective amount of astaxanthin or a related analog.

In another aspect, the present disclosure features a preparation comprising astaxanthin or a related analog prepared by the methods described herein. In an embodiment, the astaxanthin or related analog is in a more bioavailable form than astaxanthin or related analog from an algal cell in the cyst phase. In an embodiment, the preparation is formulated as a dietary supplement, e.g., human or animal dietary supplement, a nutraceutical, a natural product, a cosmetic product, or a therapeutic product.

In another aspect, the present disclosure features an astaxanthin production system comprising at least one first algal cell culture having a majority of algal cells in a green motile phase/growth phase. In an embodiment, the astaxanthin production system comprises at least one second algal cell culture having a majority of algal cells in a red motile/growth phase. In an embodiment, the astaxanthin is produced in at least one second algal cell culture by the methods of any of the preceding embodiments.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing.

FIG. 1 is a graph showing growth yield (cell abundance over time) of algal cells for different cell media, according to one embodiment.

FIG. 2 is a graph showing algal growth rate (per day) and terminal cell yield at different initial nitrate to phosphate (N:P) ratios in the growth media, according to one embodiment.

FIG. 3 are graphs of (FIG. 3A) percentage of total cells as red swimmers over time from initiation of stress conditions and at different initial nitrate to phosphate (N:P) ratios in the growth media and (FIG. 3B) temporal changes in cellular and volumetric astaxanthin content, according to one embodiment.

FIG. 4 is a series of daily photographs showing the production and accumulation of astaxanthin in carboys (left to right) treated with 10% volume addition of de-ionized water, cell free media depleted in nitrate—newly produced, and cell free media depleted in nitrate—aged, according to one embodiment.

FIG. 5 is a graph of absorption of astaxanthin in an acidic solution, pH ˜4 and approximating a fake salmon stomach, for cysts and non-cysts, according to one embodiment.

FIG. 6 is a photograph showing algal cell culture bags, according to one embodiment.

### DETAILED DESCRIPTION

The disclosure relates to methods of production and accumulation of naturally produced carotenoid compounds, such as astaxanthin. Astaxanthin has many uses as an organic colorant. One exemplary use is the enhancement of flesh color of farm-raised salmon, when provided as a dietary supplement. Astaxanthin is naturally produced by algal cells, such as Haematococcus pluvialis, during a stress-induced cell-phase transition from green vegetative cells to an immotile red cyst form.

### Algal Cells

The methods disclosed herein relate to the culture and handling of algal cells. Algal cells are eukaryotic cells that typically inhabit freshwater or saltwater aquatic locations. Algal cells may thrive in a range of temperatures, oxygen or carbon dioxide concentrations, acidity and turbidity. When cultured under the appropriate conditions, algal cells may produce light-harvesting pigments including chlorophylls, carotenoids, and phycobiliproteins, that aid in photosynthesis (biochemical reactions that use the energy of light to convert carbon dioxide and water into sugars). The light-harvesting pigments are contained in flattened, membranous sacs, called thylakoids, of the chloroplast.

In accordance with certain embodiments, the algal cells used in the methods disclosed herein may be carotenoid-producing cells. Certain algal cells may be cultured to produce carotenoids, such as astaxanthin, β-carotene, lutein, canthaxanthin, fucoxanthin, and echinenone. Carotenoid-producing algal cells include those of species Botryococcus, Chlorella, Chlorococcum, Coelastrella, Haematococcus, Dunaliella, Nanochloropsis, Scenedesmus, Spirulina, and others. Yet other carotenoid-producing algal cells that have yet to be studied are believed.

In accordance with certain embodiments, the algal cells used in the methods disclosed herein may be astaxanthin-producing cells. Exemplary astaxanthin-producing algal cells that may be used in accordance with the methods disclosed herein include Haematococcus cells. Astaxanthin is naturally produced by the stress-induced cell-phase transition of Haematococcus cells from a green vegetative state to an immotile red cyst form. Haematococcus algal cells may be readily grown in laboratory and commercial production environments. Haematococcus algal cells include Haematococcus capensis, Haematococcus carocellus, Haematococcus droebakensis, Haematococcus lacustris, Haematococcus murorum, Haematococcus pluvialis, Haematococcus thermalis, and Haematococcus zimbabwiensis.

In the culture of Haematococcus pluvialis, an exemplary Haematococcus cell, at least three cellular growth stages may be identified (1) a green motile cell stage; (2) an intermediate red, astaxanthin containing, motile stage; and (3) a final immotile red cyst stage. For example, a two-stage growth process has been described in U.S. Pat. No. 6,022,701 filed Jul. 30, 1998, titled “Procedure for large-scale production of astaxanthin from Haematococcus,” (incorporated herein by reference in its entirety for all purposes), in which green motile cells are grown to maximal density under conditions of sufficient nutrients, harvested and subjected to various stressors, mainly nutrient deprivation and high light levels, to induce astaxanthin production by transition through the red motile stage and to the final immotile cyst stage.

Various Haematococcus cell culture media for the production of astaxanthin have been studied. In Fabragas, J., et al. Optimization of culture medium for the continuous cultivation of the microalga Haematococcus pluviatilis, (Applied Microbiology and Biotechnology, 2000, 53:530-535), a single-variable optimization strategy was applied to 18 components of cell culture media to maximize the productivity of vegetative cells of H. pluvialis in semi-continuous culture. An Optimal Haematococcus Media (OHM) was determined. OHM media is conventionally used for the culture of Haematococcus cells in the production of astaxanthin. Additionally, vanadium, iodine, boron, and zinc were concluded to be non-essential for the growth of H. pluvialis.

Cells in the final cyst stage are collected for the extraction of astaxanthin. Typical extraction methods may include breaking down the cells under harsh conditions, such as heating, filtering, and wet granulation, and precipitating the astaxanthin with an acid and/or alcohol. The extraction may include addition of chemicals, such as ethyl acetate, ethanol, methanol, acetone, acetic acid, dimethyl sulfoxide (DMSO), and supercritical CO₂, among others. See, for example, Krichnavaruk, S., et al. (2008) Bioresource Technology 99(13):5556-5560. After the extraction process, an astaxanthin extract is obtained.

### Astaxanthin and Related Analogs

The methods disclosed herein relate to the production and accumulation of astaxanthin and related analogs, for example, carotenoid substances. Carotenoids are natural pigments typically produced by plants and algae species responsible for the coloring of such plants and algae. Carotenoids are terpenoids based on a structure having the formula C₄₀H₅₆.

In an embodiment, the astaxanthin or related analog (e.g., cartenoid) described herein is a compound of Formula (I):

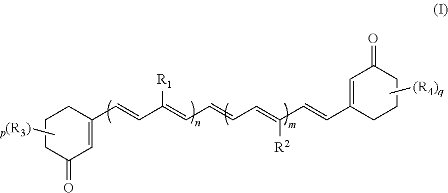

or a salt or tautomer thereof, wherein:
each of R¹ and R² is independently hydrogen, C₁-C₆ alkyl, C₂-C₆ alkenyl, or halo;
each of R³ and R⁴ is independently C₁-C₆ alkyl, C₂-C₆ alkenyl, or —OR^{A};
each of m and n is independently an integer between 1 and 10; and
each of p and q is independently an integer between 0 and 7.

In some embodiments, the compound of Formula (I) is a compound of Formula (I-a):

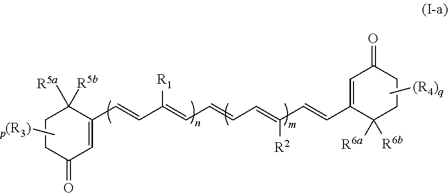

or a salt or tautomer thereof, wherein:
each of R¹ and R² is independently hydrogen, C₁-C₆ alkyl, C₂-C₆ alkenyl, or halo;
each of R³ and R⁴ is independently C₁-C₆ alkyl, C₂-C₆ alkenyl, or —OR^{A};
each of R^{5a}, R^{5b}, R^{6a}, and R^{6b} is independently C₁-C₆ alkyl, C₂-C₆ alkenyl, or —OR^{A};
each of m and n is independently an integer between 1 and 10; and
each of p and q is independently an integer between 0 and 5.

In some embodiments, the compound of Formula (I) (e.g., a compound of Formula (I) or (I-a)) is selected from astaxanthin or adonixanthin.

In an embodiment, the compound of Formula (I) (e.g., a compound of Formula (I) or (I-a)) is astaxanthin. Astaxanthin is one exemplary carotenoid that forms a red pigment. In addition to being an organic dye, astaxanthin has been shown to provide many pharmacological benefits, including anticancer, antidiabetic, anti-inflammatory, and antioxidant activities, as well as neurological, cardiovascular, ocular, and skin-protective effects. Such benefits may be conferred by the ability of astaxanthin to reduce, limit, or inhibit oxidative stress.

Astaxanthin shares metabolic and physiological functions associated with carotenoids, such as zeaxanthin, lutein, and β-carotene. However, astaxanthin has been shown to be more bioactive than these carotenoids. The bioactivity of astaxanthin may be attributed to the presence of a keto- and a hydroxyl group on each end of the molecule. Additionally, astaxanthin has unique features that support its use in promoting human health. Unlike other carotenoids, the astaxanthin molecule is not converted into vitamin A. The polar end groups of the astaxanthin molecule quench free radicals, while the double bonds of its middle segment remove high-energy electrons. The higher antioxidant activity of astaxanthin may be attributable to such features. In addition, astaxanthin is believed to preserve the integrity of cell membranes by inserting itself in membrane bilayers protecting the redox state and functional integrity of mitochondria. Furthermore, the demonstrated benefits are observed at very modest dietary intake (for example, at 2 mg-12 mg dosages) because the strong polar nature of the astaxanthin molecule optimizes the rate and extent of its absorption.

Most astaxanthin on the market is synthetic. Conventionally, natural astaxanthin is produced by altering algal cell culture conditions to induce cellular oxidative stress to transition the cells from the green motile phase to the red non-motile cyst phase. The change in culture conditions that induce cellular oxidative stress typically include an increase in light and/or depletion of nitrate through other stressors (e.g., addition of salt content) have been shown. The methods disclosed herein may be employed to produce a more bioavailable natural whole cell astaxanthin product.

### Methods of Producing Astaxanthin

Methods of producing astaxanthin are provided. The astaxanthin may be produced by algal cells in a red motile/growth phase. Furthermore, the astaxanthin contained in algal cells in a red motile/growth phase may be more bioavailable than astaxanthin contained in cells in the cyst phase. Thus, the methods may generally include culturing algal cells in a red motile/growth phase under conditions appropriate for the production and/or accumulation of astaxanthin. The methods may include culturing algal cells in a red motile/growth phase under conditions sufficient to delay transition of algal cells from a red motile/growth phase to the red non-motile cyst phase.

The duration of the culture may be dependent on cell density. In some embodiments, the methods may include culturing the algal cells in the red motile/growth phase between 2-10 days, e.g., between 3-8 days, between 4-7 days, e.g., about 2, 3, 4, 5, 6, 7, 8, 9, or 10 days (for a cell culture of about 7,000 algal cells/mL). The duration of the culture in the red motile/growth phase may be less than or equal to 6 days, for example, less than or equal to 4 days. The methods disclosed herein may produce substantially equivalent amounts of astaxanthin in 2-10 days as compared to conventional culture methods performed for a longer period of time, for example, more than 12 days. In some embodiments, the methods disclosed herein may include culturing the algal cells in the red motile phase for a duration of less than 0.75×, for example, less than 0.5×, of a reference standard, e.g., a conventional culture method.

The methods disclosed herein may increase a culture time of the red motile cells before the cells transition to the non-motile cyst phase. In some embodiments, the delay of the transition of the algal cell from the red motile/growth phase to the non-motile cyst phase lasts for 2-20 days, e.g., between 5-15 days, between 7-14 days, e.g., about 2, 3, 4, 5, 6, 7, 8, 9, 10, 11, 12, 13, 14, 15, 16, 17, 18, 19, or 20 days. For example, the delay of the transition of the algal cell from the red motile/growth phase to the cyst phase may last for at least 7 days.

The methods may comprise culturing the red motile/growth phase algal cells in a cell culture medium. In some embodiments, the cell culture medium may be effective to provide conditions appropriate for the production and/or accumulation of astaxanthin. The cell culture medium may be effective to provide conditions sufficient to delay transition from the red motile/growth phase to the cyst phase. For instance, in some embodiments, the cell culture medium may be effective to delay transition of at least about 70%, e.g., 80%, 90%, or 99% of the algal cells from the red motile/growth phase to the non-motile cyst phase after 6 days of culturing the algal cells in the red motile/growth phase. In some embodiments, the cell culture medium may be effective to delay transition of at least about 50%, e.g., 60%, 70%, 80%, 90%, or 99% of the algal cells from the red motile/growth phase to the cyst phase after 12 days of culturing the algal cells in the red motile/growth phase.

The cell culture medium may be Optimized Haematococcus Media (OHM) medium. The cell culture medium may be DY-V artificial freshwater medium, BG-11 medium, Bold's Basal Medium (BBM) (optionally, BBM+V, BBM3N), mesangial cell medium (MCM), melanocyte growth basal medium (MBM), Modified Dulbecco's Medium (MDM), melanocyte growth medium (MGM), or other known cell media. However, the cell culture medium may be a modified cell culture medium. In some embodiments, the cell culture medium may be modified by dosing the cell culture medium with a select composition of one or more trace metals.

In some embodiments, the cell culture medium comprises a plurality of trace metals. For instance, the cell culture medium may comprise zinc. The cell culture medium may comprise vanadium. The cell culture medium may comprise a plurality of trace metals, including one or more of Na₃VO₄, ZnSO₄, MnCl₂, CoCl₂, Na₂MoO₄, H₂SeO₃, and FeCl₃. Each trace metal may be present at a concentration of 0.04-10 μM, for example, 0.04-0.5 μM, 0.5-1.0 μM, 1.0-5.0 μM, or 5.0-10 μM.

In some embodiments, the cell culture medium may be substantially free of copper.

In some embodiments, the cell culture medium may be effective to reduce, limit, or inhibit production of intracellular reactive oxygen species (ROS). Under high light stress conditions, as may be used in the methods disclosed herein, algal cells often produce excess levels of intracellular ROS. Increased production of ROS may be countered by several enzymes, e.g., superoxide dismutase, ascorbate peroxidase, catalase, and others, which use trace metals, such as zinc, as metal cofactors. While not wishing to be bound by theory, it is believed the ROS-countering enzymes may provide conditions favorable for producing and/or accumulating astaxanthin by delaying transition of the algal cells to the cyst phase.

In some embodiments, the cell culture medium may be effective to increase growth rate of the algal cells cultured in a green motile/growth phase by at least 0.5-fold, e.g., 1-fold, 1.5-fold, 2-fold, 2.5-fold, or 3-fold, as compared to a reference standard. In some embodiments, the cell culture medium may be effective to increase cell biomass yield of algal cells cultured in a green motile/growth phase by at least 0.5-fold, e.g., 1-fold, 1.5-fold, 2-fold, 2.5-fold, or 3-fold, as compared to a reference standard. The reference standard may refer to a reference cell culture medium, e.g., a cell culture medium comprising copper and substantially free of zinc and vanadium trace metals.

For instance, the cell culture medium may be effective to produce a growth rate of the algal cells between about 0.2-0.5 d⁻¹, e.g., 0.3-0.4 d⁻¹. The cell culture medium may be effective to produce a cell biomass yield of algal cells cultured in the green motile/growth phase of between about 0.2-0.5 g 1⁻¹ dry weight (DW), e.g., 0.3-0.4 g 1⁻¹.

The cell culture medium may have depleted nitrate. For instance, the cell culture medium may have less than 15 mg/L, e.g., less than 10 mg/L, 8 mg/L, 6 mg/L, 5 mg/L, 4 mg/L, 3 mg/L, 2 mg/L, or 1 mg/L nitrate. In some embodiments, the cell culture medium comprises nitrate and phosphorus in a ratio of less than 19:1 (N:P), e.g., less than 18:1, 15:1, 12:1, 11:1, 10:1, 9:1, 8:1, 7:1, 6:1, 5:1, 4:1, 3:1, or 2:1. The cell culture medium may have nitrate and phosphorus in a ratio of less than 12:1 (N:P). The cell culture medium may have nitrate and phosphorous in a ratio of between about 11:1 (N:P) and 5:1 (N:P). The nitrate and phosphorus ratio may be controlled by providing a cell culture medium with depleted nitrate and/or by dosing the cell culture medium with phosphorus. While not wishing to be bound by theory, it is believed that depleted nitrate and/or a low nitrate to phosphorous ratio provides increased production of astaxanthin by the red motile algal cells.

The methods may include controlling irradiance or light intensity of the algal cells. In some embodiments, the methods may include culturing the algal cells under light intensity at an irradiance between about 30-140 μmol photons m⁻² s⁻¹, e.g., 35-110 μmol photons m⁻² s⁻¹ or 40-80 μmol photons m⁻² s⁻¹. Light intensity may be provided by a light source.

In certain embodiments, the methods may include culturing the algal cells under an irradiance of between 5-fold to 15-fold, e.g., between 8-fold to 12-fold, e.g., 8-fold, 9-fold, 10-fold, 11-fold, or 12-fold, over a reference standard, e.g., the irradiance under conventional conditions, e.g., conditions that do not increase production and/or accumulation of astaxanthin in the algal cell and/or do not delay transition of the red motile phase algal cell to the cyst phase. Thus, in some embodiments, the methods may include culturing the algal cells under light intensity at an irradiance between about 150-1400 μmol photons m⁻² s⁻¹, e.g., 175-1100 μmol photons m⁻² s⁻¹ or 200-800 μmol photons m⁻² s⁻¹.

The algal cells may be cultured under controlled light/dark conditions. For instance, the methods may include culturing the algal cells at a ratio of between about 9:15 to 15:9 light hours to dark hours (L:D), e.g., 9:15 to 10:14 (L:D), 10:14 to 13:11 (L:D), or 13:11 to 15:9 (L:D). Light hours may be controlled by providing irradiance. Dark hours may be controlled by restricting irradiance. In some embodiments, dark hours are controlled by filtering or blocking natural sunlight from the algal cells.

The methods may include controlling temperature of the algal cell culture. For instance, the methods may include controlling temperature of the culture to between about 15-28° C., e.g., 18-26° C. or 20-24° C. Temperature may be controlled by heating or cooling the algal cell culture.

The methods may include controlling pH of the algal cell culture. For instance, pH of the culture may be controlled to between about 6-8, e.g., 6, 6.5, 7, 7.5, or 8. In some embodiments, pH may be controlled by addition of an acid or a base. In some embodiments, pH may be controlled by controlling oxygen and/or carbon dioxide of the algal cell culture.

The methods may include controlling oxygen and carbon dioxide of the algal cell culture. For instance, the methods may include culturing the algal cells under controlled carbon dioxide (CO₂) conditions, e.g., between about 1-5% CO₂, e.g., 1-2% CO₂, 1% CO₂, 2% CO₂, 3% CO₂, 4% CO₂, or 5% CO₂. The controlled conditions may be provided by introducing a carbon dioxide containing gas into the cell culture. The controlled conditions may be provided by introducing an oxygen containing gas and/or other gas into the cell culture.

The methods may include agitating the algal cells. The algal cells may be agitated by introduction of a gas into the algal cell culture, e.g., an oxygen containing gas and/or a carbon dioxide containing gas. In some embodiments, the agitation may be performed by additional or alternative mechanical agitation. The agitation may be controlled to maintain viability of the algal cells.

At start up, the methods may comprise providing algal cells in the green motile/growth phase. The green motile algal cells may be inoculated to an appropriate cell abundance or density. For instance, the methods may comprise culturing the algal cells to an initial cell abundance of between about 2×10⁴ and 12×10⁴ cells ml⁻¹, e.g., 2×10⁴, 3×10⁴, 4×10⁴, 5×10⁴, 6×10⁴, 8×10⁴, 10×10⁴, or 12×10⁴ cells ml⁻¹. The methods may comprise culturing the algal cells to an initial cell density of between about 1×10³ and 1×10⁶ cells ml⁻¹, e.g., about 1×10³, 1×10⁴, 1×10⁵, or 1×10⁶ cells ml⁻¹.

The methods may comprise culturing the green motile algal cells in a cell culture medium under conditions that allow transition of the algal cell from the green motile/growth phase to the red motile/growth phase. Such conditions may include providing the appropriate cell culture media. Such conditions may additionally include providing the appropriate light levels, temperature, pH, oxygen and carbon dioxide conditions, and optional agitation of the algal cells. The conditions may be the same or different from the culture conditions previously described with respect to the red motile algal cell culture.

In some embodiments, the methods may further include culturing the algal cells (now including a majority of algal cells transitioned into the red motile algal cell stage) under conditions appropriate for astaxanthin production, as described above. In other embodiments, the methods may include separating and/or collecting algal cells transitioned into the red motile algal cell stage to form a new algal cell culture having a majority of algal cells in the red motile algal cell stage. The methods may include providing culture conditions to the new algal cell culture appropriate for production of astaxanthin, as described above. In such embodiments, at least a portion of green motile cells may be conserved as feeder cells for an initial cell culture.

The methods may comprise separating and/or collecting the algal cell in the red motile/growth phase. The red motile algal cells may be separated by centrifugation or other known cell separation methods. The astaxanthin may be harvested by drying the separated red motile algal cells. For instance, a dried astaxanthin product in powder form may be produced by lyophilization or spray drying of the separated red motile algal cells.

The methods disclosed herein may produce an astaxanthin product in a more bioavailable form than conventional methods. In certain embodiments, and as described above, the astaxanthin may be harvested without a chemical extraction. For instance, the methods may be substantially free of chemical extraction procedures.

The methods disclosed herein may be employed to produce between about 1-10% w/w of total biomass (e.g., between 1-9, 1-8, 1-7, 1-6, 1-5, 1-4, 1-3, 1-2% w/w total biomass) of astaxanthin. The total biomass of astaxanthin may be calculated as total cell mass of algal cells in the red motile/growth phase. In some embodiments, the algal cells in the red motile/growth phase include cells in a palmelloid phase.

The amount of astaxanthin produced by the methods disclosed herein may be at least about 0.1%, 0.25%, 0.5%, 0.75%, 1%, 1.25%, 1.5%, 2%, 10%, 25%, 50%, 75%, or 100% greater than an amount of astaxanthin produced by a reference standard. The reference standard may be an amount of astaxanthin produced under conventional culture conditions, e.g., conditions that do not delay transition of the algal cell from the red motile/growth phase to the cyst phase.

Astaxanthin production systems are disclosed herein. The astaxanthin production system may comprise one or more algal cell culture. Optionally, each of the one or more algal cell culture may include a majority of algal cells in a given phase. In some embodiments, the system includes at least one algal cell culture having a majority of algal cells in a green motile/growth phase. For example, the system may include 1-10 algal cell cultures having a majority of algal cells in the green motile/growth phase, for example, 1, 2, 3, 4, 5, 6, 7, 8, 9, or 10. In some embodiments, the system includes at least one algal cell culture having a majority of algal cells in a red motile/growth phase. For example, the system may include 1-40 algal cell cultures having a majority of algal cells in the red motile/growth phase, for example, 1, 2, 4, 6, 8, 10, 12, 14, 16, 18, 20, 22, 24, 26, 28, 30, 32, 34, 36, 38, or 40. The astaxanthin may be produced in the algal cell culture having a majority of algal cells in the red motile/growth phase, by the methods of any of the preceding embodiments.

In some embodiments, the system is formed of modular subsystems. Each modular subsystem may include at least one bioreactor having a majority of algal cells in the green motile/growth phase and at least one bioreactor having a majority of algal cells in the red motile/growth phase. Each modular subsystem may have 1×, 2×, 3×, 4×, 5×, 6×, 7×, 8×, 9×, or 10× red motile/growth algal cell bioreactors for each green motile/growth algal cell bioreactor. The system may be scaled up by incorporating additional modular subsystems. In some embodiments, the system may be formed of 1-100 modular subsystems, for example, 1-5, 6-10, 10-25, 25-50, 50-75, 75-100 modular subsystems, or more.

Each algal cell culture may be contained in a corresponding bioreactor. The bioreactors may be dimensioned to hold a volume of 20 L to 400 L, for example, 20 L to 60 L, 60 L to 100 L, 100 L to 150 L, 150 L to 200 L, or 200 L to 400 L. The bioreactors may be operated as a batch system. The bioreactors may be operated as a continuous system. In some embodiments, the bioreactors may be operated as a semi-continuous system.

In some embodiments, the bioreactor may be fluidly connected to a source of a gas. The gas may be an oxygen containing gas. The gas may be a carbon dioxide containing gas. The source of gas may provide agitation to the cell culture. The bioreactor may be fluidly connected to a source of cell media. The bioreactor may be fluidly connected to a source of cells. In some embodiments, a bioreactor for red motile/growth phase cells may be positioned downstream from a bioreactor for green motile/growth cells.

The system may include components for environmental control of the bioreactor, for example, sensing and control of one or more of pH, temperature, oxygen, carbon dioxide, light intensity, pressure, humidity, and other parameters. For example, the system may include a temperature control subsystem. The temperature control subsystem may comprise a temperature sensor, a heater, and/or a chiller. The system may include a pH control subsystem. The pH control subsystem may include a pH sensor and/or a source of an acid and/or base fluidly connected to one or more bioreactor. The system may include a light intensity control subsystem. The light intensity control subsystem may include a source of light. The source of light may be operably connectable to a timer. In some embodiments, the system or bioreactor includes or is formed of a light filtering or blocking material.

The system may include a controller. The controller may be operably connectable to one or more sensor. The controller may be operably connectable to one or more control subsystem, such as a temperature control subsystem, a pH control subsystem, a light intensity control subsystem, or a subsystem for control of pressure, humidity, or any other parameter. The controller may be operably connectable to one or more pump and/or baffle for directing fluids within the system. The controller may comprise or be connectable to a memory storage device, for example, a local memory storage device, a remote memory storage device, or a cloud-based memory storage device. The controller may comprise or be connectable to a user interface device, such as a computer or mobile computing device.

### Preparations Comprising Astaxanthin

Preparations comprising astaxanthin are provided. The preparations may be formulated as a dietary supplement, e.g., a pet food supplement, e.g., a fish feed supplement. The preparations may be formulated as a therapeutic formulation or dietary supplement, e.g., as an anti-inflammatory agent, e.g., as a treatment or prevention for joint pain, eye inflammation, and other inflammatory diseases or conditions. The preparations may be formulated as a therapeutic formulation or dietary supplement, e.g., as an antioxidant, e.g., as a treatment or prevention for low-density lipoprotein (LDL) oxidation, and other diseases or conditions.

The astaxanthin produced by the methods disclosed herein may be used in the formulation of one or more preparation. The astaxanthin may be in a more bioavailable form than astaxanthin produced in accordance with conventional methods. For instance, astaxanthin harvested from algal cells in the red motile phase may be in a more bioavailable form than astaxanthin harvested and extracted from an algal cell in the cyst phase.

The astaxanthin may be formulated as an organic dye. Organic dyes are coloring agents (e.g., pigments or opacifiers) incorporated as additives of suitable compositions or formed as finished products distributed for use as coloring agents. The organic dye may be classified or certified as a food-grade dye, a cosmetic-grade dye, and/or a pharmaceutical-grade dye, e.g., by an appropriate regulatory agency. The organic dye may be classified as a cytocompatible composition and/or a biocompatible composition.

The astaxanthin may be formulated in a natural product. Natural products may be or comprise products for commercial purposes, and may refer to dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, cosmetics, and spa treatments. A natural product referred to herein may comprise any one or more of the components described as a natural product to be incorporated into a composition or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

The astaxanthin may be formulated in a cosmetic product or a therapeutic product. The product may comprise astaxanthin, alone or in combination with additional agents, e.g., additional colorants or active agents, in combination with a pharmaceutically acceptable carrier and/or excipient and/or in combination with a cosmetically acceptable carrier and/or excipient. Such compositions may be used in the manufacture of a medicament.

The pharmaceutically acceptable carriers and/or excipients and/or cosmetically acceptable carriers and/or excipients are well-known to those who are skilled in the art. The choice of carrier and/or excipient will be determined in part by the particular composition, as well as by the particular method used to administer the composition. Accordingly, there is a wide variety of suitable formulations of the composition of the disclosure. The following methods and excipients are merely exemplary and are in no way limiting. Suitable carriers and excipients include solvents such as water, alcohol, and propylene glycol, solid absorbents and diluents, surface active agents, suspending agent, tableting binders, lubricants, flavors, and coloring agents. The pharmaceutically and/or cosmetically acceptable carriers can include polymers and polymer matrices. Examples of acceptable carriers include carboxymethyl cellulose, crystalline cellulose, glycerin, gum arabic, lactose, magnesium stearate, methyl cellulose, powders, saline, sodium alginate, sucrose, starch, talc and water, among others. Typically, the acceptable carrier is chemically inert to the active agents in the composition and has no detrimental side effects or toxicity under the conditions of use.

The product may be formulated for topical administration. The product may be formulated for parenteral administration, e.g., intravenous, intraperitoneal, or intramuscular. The product may be formulated as a parenteral liquid solution. The product may be formulated for enteral administration. The product may be formulated as an enteral capsule or tablet, or dietary supplement or food, e.g., food, food supplement, medical food, food additive, nutraceutical, or drink. The product may be formulated as a topical solution, oil, cream, emulsion, or gel. The product may be formulated as a shampoo, conditioner, spray, cream, gel, balm, body wash, soap, lotion, or make-up.

The preparation may be administered in conjunction with a combination treatment, e.g., a treatment authorized or commonly used in association with the relevant disease, condition, or a symptom thereof. The preparation may be formulated for administration with an anti-inflammatory agent. The preparation may be formulated for administration with an antioxidant. The preparation may be formulated for administration with a dietary supplement, e.g., a vitamin, mineral, or other food supplement.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “cosmetically acceptable” refers to a compound that is compatible with the other cosmetic ingredients of a composition and not deleterious to the subject receiving the compound or composition. A cosmetically acceptable composition or compound may be pharmaceutically acceptable. However, a cosmetically acceptable composition or compound need not be pharmaceutically acceptable.

In some embodiments, a “cosmetically acceptable” excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

As used herein, the terms “subject” or “patient” include all members of the animal kingdom including, but not limited to, vertebrates, mammals, animals (e.g., cats, dogs, horses, swine, rodents, etc.) and humans. In certain embodiments, the subject is a human. In certain embodiments, the subject is an animal, e.g., an aquatic animal, e.g., a fish, e.g., a saltwater fish or a freshwater fish.

One skilled in the art will appreciate that suitable products and methods of administering a compound of the disclosure to a subject are available, and, although more than one route can be used to administer a particular compound, a particular route can provide a more immediate and more effective reaction than another route.

### EXAMPLES

The function and advantages of these and other embodiments can be better understood from the following examples. These examples are intended to be illustrative in nature and are not considered to be limiting the scope of the invention.

### Example 1: Investigating Trace Metal Composition of Cell Culture Media

Haematococcus cells are conventionally cultured in Optimized Haematococcus Media (OHM). The inventors surprisingly discovered an improved cell culture media that increases growth rate and biomass yield of Haematococcus cells, which increases production of astaxanthin.

Starting at a cell abundance of 5×10⁴ cells ml⁻¹, the Haematococcus cells were cultured in a customized cell media (described in more detail below) under light intensity at irradiance levels of 40-80 μmol photons m⁻² s⁻¹ in a photoperiod having a 13:11 ratio of light/dark (L:D). Temperature was controlled between 20-24° C. The culture was bubbled with air supplemented with 0.5% v/v CO₂ at a rate of 0.5 LPM. Cell abundance was measured and tracked by direct cell counts.

High light stress in phytoplankton may induce the cells to produce excess intracellular reactive oxygen species (ROS). Antioxidant enzymes (including, e.g., superoxide dismutase (SOD), ascorbate peroxidase (APx), and catalase (CAT)) allow Haematococcus cells to manage oxidative stress, producing astaxanthin but not producing cysts. Such antioxidant enzymes use zinc as a metal cofactor. However, OHM does not contain zinc.

In this study, the trace metals typically found in OHM were replaced with a trace metal mix from DY-V media, which is similar, but with important differences. Specifically, the trace metals in DY-V media contain zinc and vanadium and are free of copper. Table 1 summarizes the trace metal composition of OHM media and DY-V media.

TABLE 1
Trace metal formulations tested.

| | OHM Media | DY-V Media |
|---|---|---|
| Trace Metal | (μM) | (μM) |
| Na₃VO₄•10H₂O | 0 | 0.00549 |
| ZnSO₄•7H₂O | 0 | 0.139 |
| MnCl₂•4H₂O | 4.95 | 1.01 |
| CoCl₂•6H₂O | 0.0462 | 0.0336 |
| CuSO₄•5H₂O | 0.04806 | 0 |
| Na₂MoO₄•2H₂O | 0.4959 | 0.0827 |
| H₂SeO₃ | 0.0450 | 0.023 |
| FeCl₃ | 9.988 | 3.7 |
| NaEDTA•2H₂O | 36.53 | 21.5 |

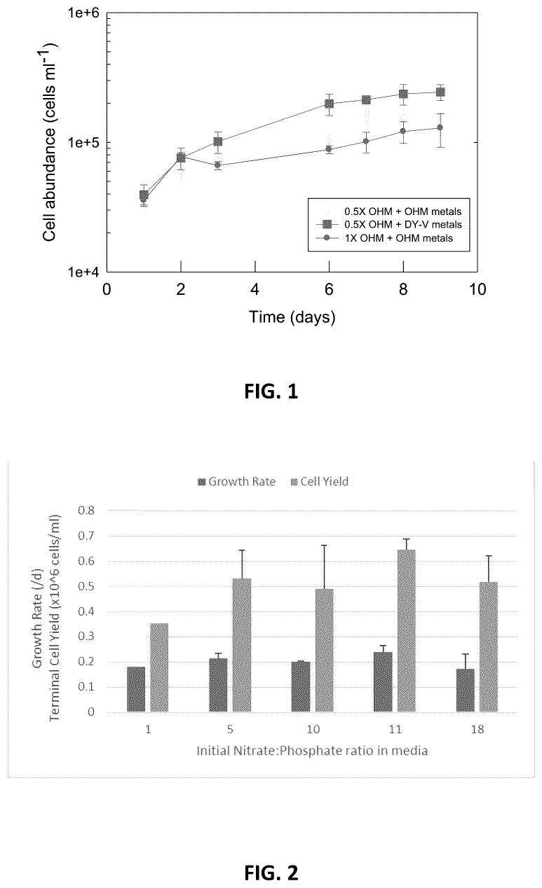

The results are shown in the graph of FIG. 1. The graph of FIG. 1 shows growth yield (cell abundance over time) as a function of different media trace metal formulations, OHM and DY-V. A base media including all elements of OHM media except trace metals, was used as a control. The 0.5× and 1× base OHM media notation refers to the relative concentrations of the base media.

As shown in the graph of FIG. 1, the DY-V trace metal culture achieved a growth rate of 0.3 d⁻¹ and a biomass yield of 0.3-0.4 g 1⁻¹ dry weight (DW) at the end of the green motile/growth stage. It was demonstrated that replacing OHM trace metals with DY-V trace metals produced a two-fold enhancement of growth rate and a greater than two-fold increase in cell biomass yield, in the tested time period. The results demonstrate a significant advancement in the Haematococcus growth phase portion of the astaxanthin production process. Haematococcus Cells are Conventionally Cultured in Media Having a High Nitrate to Phosphate (N:P) ratio. The inventors surprisingly discovered an improved cell culture media that increases production of astaxanthin without affecting growth rate and biomass yield of Haematococcus cells.

It has been previously suggested that a low N:P ratio may sustain the red motile/growth phase of H. pluvialis (Tocquin, P. et al., J. Appl. Phycol. 2012, 24, 365-373) The study concluded that a maximal and high cell density of 2×10⁶ cells ml⁻¹ was obtained, while cells remained at the vegetative and motile stage during a prolonged period of time.

In this study, nitrate to phosphate (N:P) ratio was varied by dosing the cell culture media with phosphate and not nitrate. Conventional Haemoatococcus cell culture has a N:P ratio greater than 15:1, for example, about 19:1. The Haematococcus cells were cultured in the customized cell media under conditions as described in Example 1, including the trace metals of Example 1.

The results are shown in the graph of FIG. 2. The graph of FIG. 2 shows growth rate (change in cell count over time (days)) and terminal cell yield at different initial N:P ratios in the growth media. As shown in the graph of FIG. 2, a lower N:P ratio of 5-10 does not negatively impact growth rate (observed growth rate was 0.25 d⁻¹) or cell yield. This finding is counter to the conventional belief at the time that a low N:P ratio would reduce observed growth rate. When compared to the results of Example 1, it was shown that lowering the N:P ratio alone, while controlling other aspects of the cell culture, does not adversely impact growth rate.

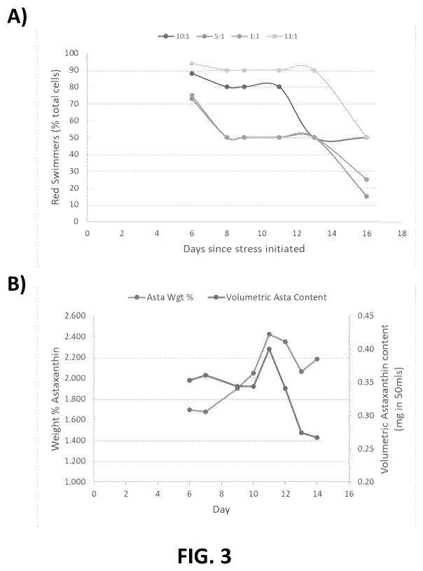

It was also shown, results in FIG. 3A, that a lower N:P ratio of 10-11 results in increased astaxanthin production. Specifically, 90% of cells or more were observed to be in the red motile/growth phase by 6 days of culture from the green transition. Under conventional culture methods, 90-100% of cells are observed to be in the red motile/growth phase after 12 days of culture (Butler et al. Biology (Basel). 2018 March; 7(1): 2). While not necessarily wishing to be bound by theory, it is believed that 90-100% of cells would be observed in the red motile/growth phase after more than 6 days of culture under the tested conditions, but before 12 days of culture. The shorter reddening time may be correlated with a significant increase in rate of production of astaxanthin. Further, it was also shown, results in FIG. 3B, that at a lower N:P of 10:11, weight percent of astaxanthin increased by ˜40% during the 6-12 day period maintaining high astaxanthin content.

Accordingly, the results demonstrate a significant advancement in production of astaxanthin by Haematococcus culture.

### Example 3: Investigating the Effect of Nitrate Depletion to Induce Cellular Astaxanthin Production

Conventionally, cellular astaxanthin is produced by altering Haematococcus culture conditions to induce cellular oxidative stress to transition the cells from the green phase to the red phase. The change in culture conditions that induce cellular oxidative stress typically include an increase in light and/or depletion of nitrate through other stressors (e.g., addition of salt content) have been used.

The inventors surprisingly discovered that cell growth media having depleted nitrate induces cellular oxidative stress and substantially increases astaxanthin production rate. A Haematococcus culture was treated to “crash” and regrow, producing higher astaxanthin content. Cell-free growth media having nitrate below 10 mg/L at 10% v/v was added to a green phase culture. Irradiance of the green culture was also increased 10-fold. Under these conditions, beginning of astaxanthin production and accumulation time was shortened to 3 days. This result was superior to a comparative culture with 5% v/v cell growth media having depleted nitrate and a control culture with deionized water, where astaxanthin production and accumulation time was 6 days. Cellular astaxanthin content was increased by 25% (% DW) over the conventional methods. The results were reproduced three times.

Accordingly, a substantial increase in astaxanthin production rate was observed. The methods advantageously reduce complexity in the handling of culture. In certain instances, the methods remove the need to dilute the parent green phase culture.

### Example 4: Investigating the Effect of Haematococcus Life Stage on Release of Astaxanthin

Astaxanthin is conventionally recovered from the cyst stage of Haematococcus by chemical extraction. Inclusion of astaxanthin-containing whole cysts into salmon feed pellets reduces bioavailability of the astaxanthin, and other valuable cellular components such as proteins and fatty acids, due to poor digestibility of the cyst wall in the salmon stomach.

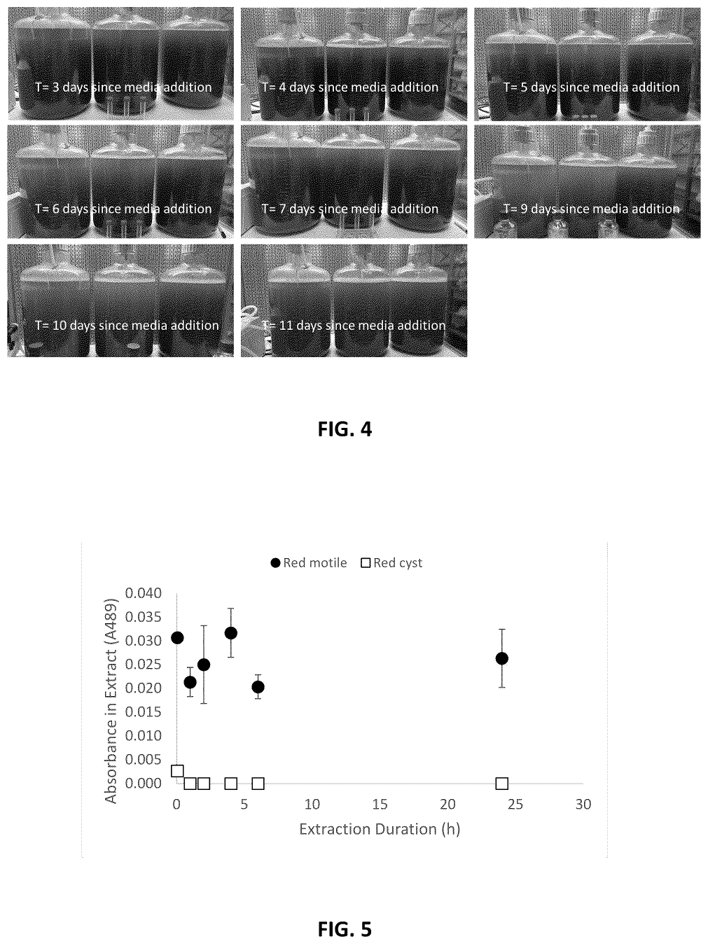

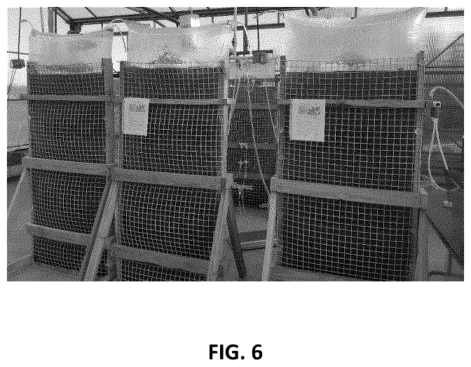

The graph of FIG. 4 shows that that astaxanthin contained in the red motile phase cells is more rapidly and quantitatively released into an acidic, pH ˜4, low oxygen solution mimicking a salmon stomach than astaxanthin contained in cysts.

### Example 5: Two-Stage Production System for Haematococcus Cell Culture

A two-stage phototrophic astaxanthin production system was designed. The system includes one 200 L green feeder cell culture and three 200 L reddening cell cultures (FIG. 5). The modular set of four cultures can be expanded as needed, for example, to 2×, 3×, 4×, 5×, 6×, or more, with further expansion when needed.

One exemplary expansion of the system includes six sets (twenty-four cultures) of cell cultures. Once running, the cell cultures are expected to be capable of producing approximately 3,000 L of red motile/growth cell culture per week. The cultures are expected to produce 0.9-1.2 kg of astaxanthin rich dried powder per week. The expected production rate is comparable to conventional production systems summarized in literature (Butler et al).

It is noted that the cell biomass yield will generally depend on natural environmental light conditions. However, the cells may be cultured under controlled light conditions, as described herein.

1. A method of producing astaxanthin or a related analog in an algal cell, wherein astaxanthin or a related analog is a compound of Formula (I): 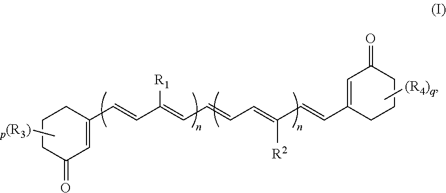
or a salt or tautomer thereof, wherein:
each of R¹ and R² is independently hydrogen, C₁-C₆ alkyl, C₂-C₆ alkenyl, or halo;
each of R³ and R⁴ is independently C₁-C₆ alkyl, C₂-C₆ alkenyl, or —OR^{A};
each of m and n is independently an integer between 1 and 10; and
each of p and q is independently an integer between 0 and 7,
the method comprising:
(i) providing the algal cell in a red motile/growth phase;
(ii) culturing the algal cell in a cell culture medium under conditions:
a. sufficient to increase production and/or accumulation of the compound of Formula (I) in the algal cell; or
b. sufficient to delay transition of the algal cell from the red motile/growth phase to the cyst phase;
thereby producing the compound of Formula (I), or a salt or tautomer thereof.

2. The method of claim 1, wherein the amount of the compound of Formula (I) produced is between about 1-10% w/w of total biomass (e.g., between 1-9, 1-8, 1-7, 1-6, 1-5, 1-4, 1-3, 1-2% w/w total biomass).

3. The method of claim 2, wherein the amount of the compound of Formula (I) produced is between about 1-5% w/w of total biomass.

4. The method of any one of claims 2-3, wherein the total biomass comprises total cell mass of algal cells in the red motile/growth phase.

5. The method of any of the preceding claims, wherein the algal cells in the red motile/growth phase includes cells in a palmelloid phase.

6. The method of any of the preceding claims, wherein the amount of the compound of Formula (I) produced is at least about 25%, 50%, 75%, or 100% greater than an amount of astaxanthin produced by a reference standard, e.g., the amount of astaxanthin produced under conditions that do not delay transition of the algal cell from the red motile/growth phase to the cyst phase.

7. The method of any of the preceding claims, wherein a duration of the culturing of the algal cell in the red motile/growth phase is between 2-10 days, e.g., between 3-8 days, between 4-7 days, e.g., about 2, 3, 4, 5, 6, 7, 8, 9, or 10 days for a cell culture of about 7,000 algal cells/mL.

8. The method of claim 7, wherein a duration of the culturing of the algal cell in the red motile/growth phase is less than or equal to 6 days.

9. The method of claim 8, wherein a duration of the culturing of the algal cell in the red motile/growth phase is less than or equal to 4 days.

10. The method of any of the preceding claims, wherein the delay of the transition of the algal cell from the red motile/growth phase to the cyst phase lasts for 2-14 days, e.g., between 5-14 days, between 7-14 days, e.g., about 2, 3, 4, 5, 6, 7, 8, 9, 10, 11, 12, 13, or 14 days.

11. The method of claim 10, wherein the delay of the transition of the algal cell from the red motile/growth phase to the cyst phase lasts for at least 7 days.

12. The method of any of the preceding claims, comprising culturing the algal cell in a green motile/growth phase in a cell culture medium having depleted nitrate, e.g., less than 10 mg/L, 8 mg/L, 6 mg/L, 5 mg/L, 4 mg/L, 3 mg/L, 2 mg/L, or 1 mg/L.

13. The method of any of the preceding claims, comprising culturing the algal cell in a green motile/growth phase under an irradiance of between 5-fold to 15-fold, e.g., between 8-fold to 12-fold, e.g., 8-fold, 9-fold, 10-fold, 11-fold, or 12-fold, over a reference standard, e.g., the irradiance under conditions that do not increase production and/or accumulation of the compound of Formula (I) in the algal cell.

14. The method of any of the preceding claims, wherein the cell culture medium comprises a plurality of trace metals.

15. The method of any of the preceding claims, wherein the cell culture medium comprises zinc.

16. The method of any of the preceding claims, wherein the cell culture medium comprises vanadium.

17. The method of any of the preceding claims, wherein the cell culture medium comprises a plurality of trace metals comprising Na₃VO₄, ZnSO₄, MnCl₂, CoCl₂, Na₂MoO₄, H₂SeO₃, and FeCl₃.

18. The method of any of the preceding claims, wherein the cell culture medium is substantially free of copper.

19. The method of any of the preceding claims, wherein the cell culture medium is effective to increase growth rate of the algal cells cultured in a green motile/growth phase by at least 0.5-fold, e.g., 1-fold, 1.5-fold, 2-fold, 2.5-fold, or 3-fold, as compared to a reference standard, e.g., a reference cell culture medium, e.g., a cell culture medium comprising copper and substantially free of zinc and vanadium trace metals.

20. The method of any of the preceding claims, wherein the cell culture medium is effective to increase cell biomass yield of algal cells cultured in the green motile/growth phase by at least 1.5-fold, 2-fold, 2.5-fold, or 3-fold, as compared to a reference standard, e.g., a reference cell culture medium, e.g., a cell culture medium comprising copper and being substantially free of zinc and vanadium trace metals.

21. The method of any of the preceding claims, wherein the cell culture medium is effective to produce a growth rate of the algal cells between about 0.2-0.5 d⁻¹, e.g., 0.3-0.4 d⁻¹.

22. The method of any of the preceding claims, wherein the cell culture medium is effective to produce a cell biomass yield of algal cells cultured in the green motile/growth phase of between about 0.2-0.5 g 1⁻¹ dry weight (DW), e.g., 0.3-0.4 g 1⁻¹.

23. The method of any of the preceding claims, wherein the cell culture medium is effective to reduce, limit, or inhibit production of intracellular reactive oxygen species (ROS).

24. The method of any of the preceding claims, wherein the cell culture medium comprises nitrate and phosphorus in a ratio of less than 19:1 (N:P), e.g., less than 18:1, 15:1, 12:1, 11:1, 10:1, 9:1, 8:1, 7:1, 6:1, 5:1, 4:1, 3:1, or 2:1.

25. The method of any of the preceding claims, wherein the cell culture medium comprises nitrate and phosphorus in a ratio of less than 12:1 (N:P).

26. The method of any of the preceding claims, wherein the cell culture medium comprises nitrate and phosphorous in a ratio of between about 11:1 (N:P) and 5:1 (N:P).

27. The method of any of the preceding claims, wherein the cell culture medium is effective to delay transition of at least about 70%, e.g., 80%, 90%, or 99% of the algal cells from the red motile/growth phase to the cyst phase after 6 days of culturing the algal cell in the red motile/growth phase.

28. The method of any of the preceding claims, wherein the cell culture medium is effective to delay transition of at least about 50%, e.g., 60%, 70%, 80%, 90%, or 99% of the algal cells from the red motile/growth phase to the cyst phase after 12 days of culturing the algal cell in the red motile/growth phase.

29. The method of any of the preceding claims, comprising controlling temperature of the culture to between about 15-28° C., e.g., 18-26° C. or 20-24° C.

30. The method of any of the preceding claims, comprising culturing the algal cell under light intensity at an irradiance between about 150-1400 μmol photons m⁻² s⁻¹, e.g., 175-1100 μmol photons m⁻² s⁻¹ or 200-800 μmol photons m⁻² s⁻¹.

31. The method of any of the preceding claims, comprising culturing the algal cell under light intensity at an irradiance between about 30-140 μmol photons m⁻² s⁻¹, e.g., 35-110 μmol photons m⁻² s⁻¹ or 40-80 μmol photons m⁻² s⁻¹.

32. The method of any of the preceding claims, comprising culturing the algal cell in light/dark conditions at a ratio of between about 9:15 to 15:9 (L:D), e.g., 9:15 to 10:14 (L:D), 10:14 to 13:11 (L:D), or 13:11 to 15:9 (L:D).

33. The method of any of the preceding claims, comprising controlling pH of the culture to between about 6-8, e.g., 6, 6.5, 7, 7.5, or 8.

34. The method of any of the preceding claims, wherein the algal cell culture is physically agitated.

35. The method of any of the preceding claims, comprising culturing the algal cell at an initial cell density of between about 1×10³ and 1×10⁶ cells ml⁻¹, e.g., about 1×10³, 1×10⁴, 1×10⁵, or 1×10⁶ cells ml⁻¹.

36. The method of any of the preceding claims, comprising culturing the algal cell at an initial cell abundance of between about 2×10⁴ and 12×10⁴ cells ml⁻¹, e.g., 2×10⁴, 3×10⁴, 4×10⁴, 5×10⁴, 6×10⁴, 8×10⁴, 10×10⁴, or 12×10⁴ cells ml⁻¹.

37. The method of any of the preceding claims, comprising culturing the algal cell under controlled carbon dioxide (CO₂) conditions, e.g., between about 1-5% CO₂, e.g., 1-2% CO₂, 1% CO₂, 2% CO₂, 3% CO₂, 4% CO₂, or 5% CO₂.

38. The method of any one of the preceding claims, further comprising separating/collecting the algal cell in the red motile/growth phase.

39. The method of any of the preceding claims, wherein the algal cell is a Haematococcus cell.

40. The method of any of the preceding claims, wherein the algal cell is selected from the group consisting of Haematococcus capensis, Haematococcus carocellus, Haematococcus droebakensis, Haematococcus lacustris, Haematococcus murorum, Haematococcus pluvialis, Haematococcus thermalis, and Haematococcus zimbabwiensis.

41. The method of any of the preceding claims, further comprising:
providing the algal cell in the green motile/growth phase;
culturing the algal cell in a cell culture medium under conditions that allow transition of the algal cell from the green motile/growth phase to the red motile/growth phase; and
separating/collecting the algal cell in the red motile/growth phase to provide the algal cell in the red motile/growth phase for production of the compound of Formula (I).

42. An algal cell in the red motile/growth phase cultured by the methods of any of the preceding claims.

43. The algal cell of claim 42, comprising an effective amount of the compound of Formula (I).

44. A preparation comprising the compound of Formula (I) prepared by the methods of any of the preceding claims.

45. The preparation of claim 44, wherein the compound of Formula (I) is in a more bioavailable form than astaxanthin from an algal cell in the cyst phase.

46. The preparation of any of claims 44-45, formulated as a dietary supplement, e.g., human or animal dietary supplement, a nutraceutical, a natural product, a cosmetic product, or a therapeutic product.

47. An production system for astaxanthin or a related analog comprising:
at least one first algal cell culture having a majority of algal cells in a green motile phase/growth phase;
at least one second algal cell culture having a majority of algal cells in a red motile/growth phase,
wherein the compound of Formula (I) is produced in at least one second algal cell culture by the methods of any of the preceding claims.

48. The method of any one of the preceding claims, wherein the compound of Formula (I) is a compound of Formula (I-a): 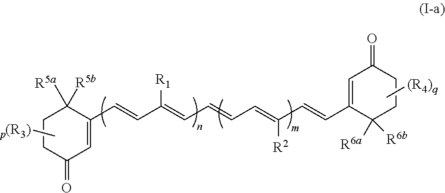
or a salt or tautomer thereof, wherein:
each of R¹ and R² is independently hydrogen, C₁-C₆ alkyl, C₂-C₆ alkenyl, and halo;
each of R³ and R⁴ is independently C₁-C₆ alkyl, C₂-C₆ alkenyl, or —OR^{A};
each of R^{5a}, R^{5b}, R^{6a}, and R^{6b} is independently C₁-C₆ alkyl, C₂-C₆ alkenyl, or —OR^{A};
each of m and n is independently an integer between 1 and 10; and
each of p and q is independently an integer between 0 and 5.

49. The method of any one of the preceding claims, wherein the compound of Formula (I) is selected from astaxanthin and adonixanthin.

50. The method of any one of the preceding claims, wherein the compound of Formula (I) is astaxanthin.
